(19) United States
(12) Patent Application Publication
(10) Pub. No.: US 2003/0170744 A1
(43) Pub. Date: Sep. 11, 2003

(54) Multiplexed analysis of cellular responses using endogenous reporter genes

(76) Inventor: Oren E. Beske, Belmont, CA (US)

Correspondence Address: KOLISCH HARTWELL, P.C., 520 S.W. YAMHILL STREET, SUITE 200, PORTLAND, OR, 97204, US

(21) Appl. No.: 10/382,796

(22) Filed: Mar. 5, 2003

Related U.S. Application Data
(60) Provisional application No. 60/362,055, filed on Mar. 5, 2002.

(51) Int. Cl. C12Q 1/00; G01N 33/53; G01N 33/567

(52) U.S. Cl. 435/7.2; 435/4

(57) ABSTRACT

System, including methods and compositions, for multiplexed analysis of cellular responses using endogenous, response-selective reporter genes.

### CROSS-REFERENCE TO PRIORITY APPLICATION

This application claims the benefit under 35 U.S.C. §119(e) of U.S. Provisional Patent Application Serial No. 60/362,055, filed Mar. 5, 2002, which is incorporated herein by reference in its entirety for all purposes.

### CROSS-REFERENCES TO RELATED APPLICATIONS

This application incorporates by reference in their entirety for all purposes the following U.S. patent applications: Ser. No. 09/549,970, filed Apr. 14, 2000; Ser. No. 09/694,077, filed Oct. 19, 2000; Ser. No. 10/119,814, filed Apr. 9, 2002; Ser. No. 10/120,900, filed Apr. 10, 2002; Ser. No. 10/186,219, filed Jun. 27, 2002; Ser. No. 10/238,914, filed Sep. 9, 2002; Ser. No. 10/273,605, filed Oct. 18, 2002; Ser. No. 10/282,904, filed Oct. 28, 2002; and Ser. No. 10/282,940, filed Oct. 28, 2002.

This application also incorporates by reference in their entirety for all purposes the following U.S. provisional patent applications: Serial No. 60/362,001, filed Mar. 5, 2002; Serial No. 60/362,238, filed Mar. 5, 2002; Serial No. 60/370,313, filed Apr. 4, 2002; Serial No. 60/383,091, filed May 23, 2002; Serial No. 60/383,092, filed May 23, 2002; Serial No. 60/413,407, filed Sep. 24, 2002; Serial No. 60/413,675, filed Sep. 24, 2002; and Serial No. 60/426,633, filed Nov. 14, 2002.

This application also incorporates by reference in their entirety for all purposes the following PCT patent applications: Serial No. PCT/US00/10181, filed Apr. 14, 2000, and published as Publication No. WO 00/63419 on Oct. 26, 2000; Serial No. PCT/US01/51413, filed Oct. 18, 2001, and published as Publication No. WO 02/37944, May 16, 2002; Serial No. PCT/US02/33350, filed Oct. 18, 2002; and Serial No. PCT/US02/34699, filed Oct. 28, 2002.

### FIELD OF THE INVENTION

The invention relates to multiplexed analysis of cellular responses. More specifically, the invention relates to multiplexed analysis of cellular responses using endogenous, response-selective reporter genes.

### BACKGROUND

Reporter genes are used in high-throughput cell assays as indicators of cellular responses to drug candidates. RNA or protein expressed from the reporter genes is measured to define a level of expression of the reporter genes and to infer, for example, activities of cell receptors. Reporter genes may be exogenous, provided by foreign nucleic acids, or endogenous, provided by native genes.

Exogenous reporter genes have gained wide popularity for both high-throughput screening and basic research. These reporter genes generally encode quickly and easily quantifiable proteins, for example, enzymes, such as chloramphenicol acetyltransferase or beta-galactosidase, or fluorescent proteins, such as green fluorescent protein. Expression of such proteins may be controlled by regulatory sequences in the reporter genes, which mimic, albeit imperfectly, regulation of an endogenous gene or set of genes. Exogenous reporter genes may be used as targets that indicate the activity of cellular responses, such as specific signaling proteins or particular response pathways. For example, the cells may be engineered to express a specific receptor of interest and to include a target reporter gene that responds to the receptor. With these engineered cells, receptor activity in response to test compounds can be assessed by monitoring expression of the reporter gene. This approach may be used to identify natural or synthetic, activating or inhibiting ligands for the receptor in screens of compounds, thus providing potential drugs for in vivo use.

Despite their prevalence, exogenous reporter systems may have a number of disadvantages for analysis of cellular responses. These disadvantages may include inefficient introduction into cells, limited ability to assay a wide variety of cells efficiently, modification of cells during and/or after introduction, and/or inability to distinguish responses produced by related receptors, as described below.

Exogenous reporter genes may be undesirable because they are difficult to introduce efficiently into many cell types, particularly primary cells. Thus, only a fraction of cells in an assay may measure reporter gene expression, resulting in a reduced reporter gene signal, or cells carrying the exogenous reporter gene may need to be separated from other cells prior to an assay. Such separation may be time-consuming and expensive, and may modify the biological properties of the cells. Accordingly, assays with exogenous reporter genes often employ cell types that efficiently take up exogenous reporter genes and grow rapidly, but may be poor models of normal cell physiology.

Exogenous reporter genes also may be undesirable because they are only crude indicators of endogenous gene activities. These reporter genes lack many of the regulatory sequences or structures that control endogenous gene expression, such as regulatory sequences carried in introns, coding exons, 3′ untranslated regions, and/or other more distantly spaced or context-dependent regulatory regions or structural domains. As a result, exogenous reporter genes often respond promiscuously to the activities of many structurally related receptors. Thus, even with distinguishable reporter gene outputs, related receptors often cannot be analyzed together on reporter genes having identical regulatory regions in a single cell. Instead, the related receptors may need to be carried by separate cells to distinguish their effects on the reporter genes. Furthermore, the receptors may need to be overexpressed to increase their activity on the introduced exogenous reporter genes relative to endogenous receptors. Overexpression of receptors further distances the analysis from normal cell physiology. Therefore, some drugs identified by assays using exogenous reporter genes may have no therapeutic value.

In summary, due to the many potential disadvantages of exogenous reporter genes, endogenous reporter systems are needed to perform multiplexed analyses of cellular responses.

### SUMMARY OF THE INVENTION

The invention provides a system, including methods and compositions, for multiplexed analysis of cellular responses using endogenous, response-selective reporter genes.

### BRIEF DESCRIPTION OF THE DRAWINGS

FIG. 1 is a flowchart of a method for multiplexed analysis of cellular responses using expression of endogenous reporter genes to characterize a candidate modulator relative to a set of cellular responses, in accordance with aspects of the invention

FIG. 2 is a schematic view of an exemplary implementation of the method of FIG. 1 in which the effect of a candidate modulator is compared to a plurality of cellular responses, in accordance with aspects of the invention.

FIG. 3 is a schematic view of a variation of the implementation of FIG. 2 in which the candidate modulator is analyzed for its ability to modify each of the cellular responses, in accordance with aspects of the invention.

FIG. 4 is a schematic view of a mixed effect on the cellular responses that may be obtained from the variation of FIG. 3.

FIG. 5 is a schematic view of another mixed effect on the cellular responses that may be obtained from the variation of FIG. 3.

FIG. 6 is a schematic view of another exemplary implementation of the method of FIG. 1 in which the effect of a candidate modulator is compared to a plurality of cellular responses produced in different cell types, in accordance with aspects of the invention.

### DETAILED DESCRIPTION

The invention provides a system, including methods and compositions, for multiplexed analysis of cellular responses. The analysis may measure the level of expression of endogenous reporter genes, as protein, RNA, or enzyme activity, among others. The endogenous reporter genes are response-selective, meaning that the level of expression of each reporter gene changes substantially for only one of the responses of interest in cells. Accordingly, the levels of expression of the endogenous reporter genes may serve as a code to relate any candidate modulator to each of the cellular responses of interest. When each candidate modulator is a drug candidate, the system described herein may be used to screen the drug candidates for their effects on each of the cellular responses, such as signal transduction responses initiated by different ligands. Thus, the system may identify new agonists or antagonists of cellular responses with improved response specificity, increased physiological relevance, and/or with less manipulation of cells.

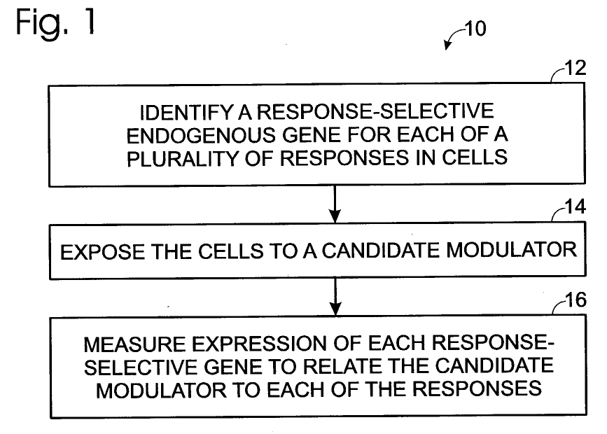

FIG. 1 shows a flowchart of a method 10 for multiplexed analysis of the effect of a candidate modulator on a plurality of cellular responses using endogenous reporter genes. Method 10 may include an identifying step, shown at 12, an exposing step, shown at 14, and a measuring step, shown at 16. The steps of exposing and measuring may be repeated a plurality of times to screen a plurality of candidate modulators, such as a library of compounds in a drug screen.

Method 10 may identify or define one or more response-selective endogenous genes for each of a plurality of responses of interest in cells, shown at 12. The endogenous genes are termed “response-selective” because the genes show a substantial change in expression in correspondence with one of the responses of interest, but not the other responses of interest. The substantial change may be any measurable increase or decrease in expression brought about by the response, such as at least about 1.5-fold, 2-fold, 5-fold, or 10-fold, among others. Each response-selective endogenous gene may be termed a reporter gene, because changes in expression of the gene may be related to and thus may report an aspect of one of the responses. Cellular responses, endogenous reporter genes, and cells are described in more detail below in Sections I, II, and III, respectively.

Method 10 may expose the cells to a candidate modulator, shown at 14. The candidate modulator may be chemical, physical, and/or biological. Exposure may include addition of the candidate modulator to the cells in a compartment and incubation for any suitable length of time. Modulators are described below in more detail in Section IV.

Method 10 may measure expression of each response-selective gene to relate any effect of the candidate modulator to each of the cellular responses, as shown at 16. Measurement may determine a level of expression for each reporter gene. The level may be a level of RNA, protein, and/or activity produced from expression of the reporter gene. Changes in expression of a reporter as a result of exposure to the candidate modulator (relative to no exposure) may be used to relate the effect of the modulator to a particular one of the responses. Accordingly, the potency and specificity of the candidate modulator for each of the responses may be determined. Measurement of expression levels is described in more detail below in Section V.

Further aspects of the invention are described in the following sections: (I) cellular responses, (II) reporter genes, (III) cells, (IV) modulators, (V) measurement of expression levels, and (VI) examples.

I. Cellular Responses

The system described herein measures changes in endogenous reporter genes produced by cellular responses. A cellular response may be any measurable change in the state of a cell, generally as a result of a particular treatment of the cell or as a spontaneous or adaptive change of the cell. The measurable change may be a phenotypic change and/or a biochemical change, among others. Exemplary phenotypic changes may include differentiation, transformation, anchorage independence, a switch to growth from a quiescent state, a switch to quiescence from a growth state, activation of a signal transduction pathway, inhibition of a signal transduction pathway, response to heat shock or DNA damage, changes in transport or distribution of cellular components, etc. Exemplary biochemical changes may include changes in level, structure, or partnership of particular proteins, RNAs, lipids, carbohydrates, metabolites, and/or other cellular constituents. Each cellular response should include changes in endogenous gene expression so that endogenous reporter genes can be identified.

Any suitable treatment may produce a cellular response. The treatment may be chemical, physical, and/or biological. Exemplary chemical treatments may include exposure to a ligand (such as a peptide growth factor, a steroid hormone, etc.), change of ionic strength and/or pH, changes in the extracellular matrix, and/or the like. Exemplary physical treatments may include a change in temperature or pressure, or exposure to an electric or magnetic field, light (i.e., electromagnetic radiation), or radiation (e.g., alpha, beta, and/or gamma emitters), among others. Exemplary biological treatments may include apposition to another cell, infection with a virus, transfection, and/or the like.

II. Reporter Genes

Cellular responses may be measured using endogenous reporter genes. Endogenous reporter genes generally comprise any genes showing response-selective changes in expression. In addition, endogenous reporter genes are native genes, that is, at least the majority of each gene is included naturally in a cell's genome, rather than introduced artificially through human manipulation. Accordingly, an endogenous gene has arrived in the genome of a cell at least substantially by natural processes such as genome evolution, natural selection, natural infection, etc., and not by transfection or other modes of cell engineering.

Endogenous reporter genes may be identified or defined before they are used in multiplexed analysis of cellular responses. Such reporter genes may be identified by separately initiating different cellular responses of interest in cells, for example, by treating the cells with different ligands of interest. Changes in gene expression associated with each response then may be measured to identify genes that are selectively responsive to less than all of the different cellular responses, generally only one of the cellular responses.

The cellular response may be a change in activity of a ligand-response pathway. Such a response pathway generally comprises any selective response of an endogenous reporter gene to a ligand. A selective response means that expression of the reporter gene responds to exposure of only one ligand or subset of ligands out of a defined set of known ligands of interest. For example, expression of individual genes from a cell population may be measured without ligand treatment, as a control, and with ligand treatment, separately for each ligand or each set of ligands. Thus, if there are three ligands, L1, L2, and L3, suitable reporter genes for multiplexed assays on the L1-L3 response pathways may change substantially in their expression level only in the presence of one of L1-L3. In other embodiments, reporter genes may be identified for two, four, or more ligand-response pathways or cellular responses.

Suitable reporter genes may be identified from among candidate genes based not only on selectivity of response but also on the magnitude and polarity of a change in expression. Suitable magnitudes and polarities may be based at least partially on whether each response is produced in all of the cells or in only a subsets of the cells, such as in a mixture of different cell types. When substantially all of the cells produce each response of interest, such as in a clonal cell population, a suitable reporter gene may show a response-selective increase or decrease in expression, and a smaller change in expression may be detectable. However, when only a subset of the cells provide each cellular response, such as in a mixture of two or more different cell types, suitable reporter genes may need to have a greater magnitude of change with their corresponding responses. In addition, such suitable reporter genes may be expressed substantially in only in a subset of the mixture, both in a control state before any response and during and/or after the response. Some candidate reporter genes may not be suitable for multiplexed assays in this cell mixture because the magnitude of changes in expression of the reporter genes in responsive cells are diluted and thus diminished by unresponsive cells in the cell mixture. Some multiplexed assays with either a clonal or mixed cell population may benefit from identification of reporter genes that change with their corresponding cellular responses by completely shutting off expression or by turning on expression from a silent state, as an averaged response from the cell population.

Suitable endogenous reporter genes may be identified by any appropriate method that quantifies RNA or protein, for example, as described below, in Section V.

III. Cells

The system described herein uses cells to provide endogenous reporter genes and cellular responses. Any suitable cells may be used. The cells may be a single type of cells or a mixture of two or more types of cells. Accordingly, the cells may be a substantially clonal cell population, or a set of two or more distinct cell types. When two or more cell types are used, each endogenous reporter gene may be selectively responsive in only one type of cells so that each type of cells provides a cell-type restricted response. However, in some embodiments, a corresponding gene sequence for the reporter gene may be present in the genomes of the other types. For example, a cell mixture may be used that includes human cells from heart, kidney, and liver to provide heart-, kidney-, and liver-specific responses. A reporter gene for the heart-specific response generally is included in all human cells. However, this reporter gene may show no change in expression level in the kidney and liver cells in correspondence with the kidney- and liver-specific responses.

Suitable cells may include any type of cell or protein-expressing organelle, or combination thereof. Exemplary cells include primary cells, established cell lines, patient samples, and/or cells transfected with an expression vector encoding a protein of interest, such as a receptor(s), among others. In some embodiments, cells or subsets of cells may express a transcription factor(s) with an engineered DNA binding specificity, such as a zinc finger protein engineered to bind to a preselected target. Methods for making and using such engineered zinc finger proteins are disclosed in U.S. Pat. No. 6,453,242, issued Sep. 17, 2002, which is incorporated herein by reference. Suitable receptors that may be expressed from endogenous or exogenous genes may include any subcellular components capable of binding to a corresponding ligand, including cell-surface receptors (such as G-protein coupled receptors or cytokine receptors, among others), cytoplasmic receptors, and nuclear hormone receptors (such as receptors for steroid hormones, thyroid hormones, and/or retinoids, among others). Cells may be modified, by transfection of nucleic acids or proteins, to include or express any proteins capable of affecting expression of endogenous reporter genes or modifying cellular responses. Such proteins may include kinases, phosphatases, receptors, DNA binding proteins, RNA binding proteins, acetylases, deacetylases, methylases, demethylases, helicases, topoisomerases, transcription factors or cofactors, and transcription inhibitors, among others. Exemplary organelles include mitochondria and/or chloroplasts, among others. Other exemplary cells that may be suitable are described in more detail in the patent applications identified above under Cross-References, which are incorporated herein by reference, particularly Ser. No. 10/120,900, filed Apr. 10, 2002.

IV. Modulators

Cells including endogenous reporter genes may be exposed to modulators. Modulators generally include any chemical or biological material, or physical treatment, that is known or suspected to affect one or more of the cellular responses. In some embodiments, the cells may be exposed to candidate modulators. Candidate modulators may include any potential modulator being tested for its effect on each of the cellular responses.

Chemical modulators generally comprise any chemical material(s), such as an element, a salt, a molecule, a polymer, a mixture, an extract, and/or the like. Exemplary chemical modulators may include proteins, peptides, nucleic acids, lipids, carbohydrates, small compounds, etc. A chemical modulator may be a ligand that binds to a corresponding receptor(s). Ligands may be known ligands, for example, used to initiate cellular responses. Alternatively, ligands may be candidate ligands, that is, ligands added during a multiplexed assay to determine their effects on each of the cellular responses being analyzed in the assay. Known and candidate ligands each may act as agonists and/or antagonists of receptor function, and thus may activate and/or repress expression of endogenous reporter genes.

A multiplexed assay may be conducted using one or more candidate modulators, with or without predefined initiators of the cellular responses. For example, a single candidate modulator may be tested alone for its ability to produce each of the cellular responses of interest. Alternatively, two or more candidate modulators may be tested together, but without the predefined initiators, for their ability to produce each cellular response, especially in assays in which a measured or desired effect of candidate modulators is uncommon. In other embodiments, initiators of the cellular responses may be combined with one or more candidate modulators. In these embodiments, the assay may measure the ability of each candidate modulator to alter the cellular responses, that is, enhance, diminish, or abolish each response.

Further aspects of modulators, particularly chemical, physical, and biological modulators, are described in more detail in the patent applications identified above under Cross-References, which are incorporated herein by reference, particularly Ser. No. 10/120,900, filed Apr. 10, 2002.

V. Measurement of Expression Levels

The level of expression of an endogenous reporter gene may be measured and utilized as a reporter signal. The level of expression or reporter signal generally comprises the presence, absence, quantity (absolute (e.g., mass or number) or relative (e.g., concentration)), and/or activity (such as enzyme activity) of an RNA or protein expressed from the endogenous reporter gene. Any of these values may be measured from a cell extract and/or in whole cells, using any suitable method.

Exemplary methods for RNA quantification include hybridization, for example, to a solid support, in solution phase, and/or to cells in situ, among others. Methods for RNA hybridization to a solid support include nucleic acid arrays, microarrays, or Northern blots. Methods for RNA hybridization in solution phase include PCR or related amplification methods, nuclease protection assays, and/or primer extension, among others, with each method detecting nucleic acid (e.g., using energy transfer probes). In some embodiments, a combination of solid phase and solution phase hybridization may be used. For example, U.S. Pat. No. 6,238,869 to Kris et al., which is incorporated by reference herein, describes nuclease protection in the presence of gene-specific probes, followed by hybridization of the probes to an array formed with a set of immobilized target sequences. This combined approach can be conducted in a microplate format and may be suitable for high-throughput multiplexed analysis of a set of endogenous reporter genes, such as about 2-100, 4-50, or 10-20 genes, among others. In some embodiments, the analysis may be repeated a plurality of times with different candidate modulators in different wells of a microplate(s). Por example, the cells may be exposed to different candidate modulators in different microplate wells. Expression levels may be measured in these wells or in other microplate wells.

Exemplary methods for protein quantification include immunological methods, electrophoresis, chromatography, affinity adsorption, mass spectrometry, and/or immunohistochemical staining, among others. Immunological methods may include ELISA assays or other suitable antibody-based measurements. Electrophoretic methods include one- or two-dimensional gel electrophoresis, followed by general or specific protein detection, for example, by antibody staining. Chromatography and affinity methods include affinity matrices, size or ion exchange resins, high-pressure liquid chromatography and/or so on. Immunohistochemical staining includes antibody labeling of cells and tissues.

VI. EXAMPLES

The following examples describe selected aspects and embodiments of the invention, including methods for multiplexed analysis of the effect of candidate modulators on a set of cellular responses using endogenous reporters. These examples are included for illustration and are not intended to limit or define the entire scope of the invention.

### Example 1

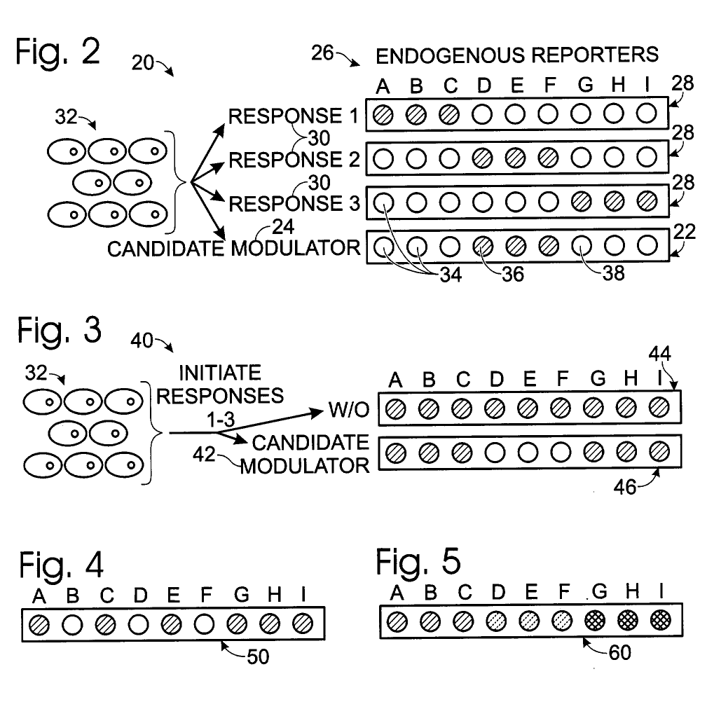

This example describes exemplary implementations of method 10 of FIG. 1 to analyze candidate modulators; see FIGS. 2-6.

FIG. 2 shows a schematic view of an exemplary implementation 20 of method 10. Implementation 20 may compare the effect 22 of a candidate modulator 24 on endogenous reporter genes 26 to the effects 28 produced by a plurality of cellular responses 30 in cells 32. The modulator effect 22 and the response effects 28 may be represented by measured expression levels 34 of endogenous reporter genes 26, which are shown here as genes A-I. Expression levels corresponding to a change (measurable increase or decrease) or no change (relative to before the response) are shown at 36 (hatched) and 38 (unhatched), respectively. Accordingly, genes A-C are selective for response “1,” genes D-F for response “2,” and genes G-I for response “3.” In addition, the candidate modulator tested here produces changes in expression levels of the endogenous reporter genes that are similar to response “2.” Accordingly, this candidate modulator may be considered as a selective agonist for this response relative to the other responses analyzed.

FIG. 3 shows a schematic view of a variation 40 of implementation 20. In variation 40, another candidate modulator 42 may be analyzed for its ability to modify each of the cellular responses by initiating all responses in cells 32, with or without the modulator. When the responses are initiated in the absence of modulator 42, shown at 44, all responses are produced together as indicated by a change in expression level of each reporter gene. However, when the responses are initiated in the presence of the modulator, shown at 46, the expression levels of genes D-F, corresponding to response “2” (see FIG. 2) are at their original, unchanged levels. Accordingly, modulator 42 is an antagonist that selectively antagonizes response “2” relative to the other responses.

FIG. 4 shows a schematic view of an alternative, mixed effect 50 on the cellular responses that may be obtained from variation 40. Here, the candidate modulator shows no effect on response “3” as indicated by no effect on expression of genes G-I, but affects expression of gene B of response “1” and expression of genes D and F of response “2.” Accordingly, the candidate modulator lacks specificity in this case.

FIG. 5 shows a schematic view of another type of mixed effect 60 on the cellular responses that may be obtained from variation 40. Here, the modulator produces no change in the expression levels of genes A-C, a decrease in the change in expression levels of genes D-F, and an increase in the change in expression levels of genes G-I. Therefore, in this case the modulator is acting as an antagonist of response “2,” and as an agonist of response “3.”

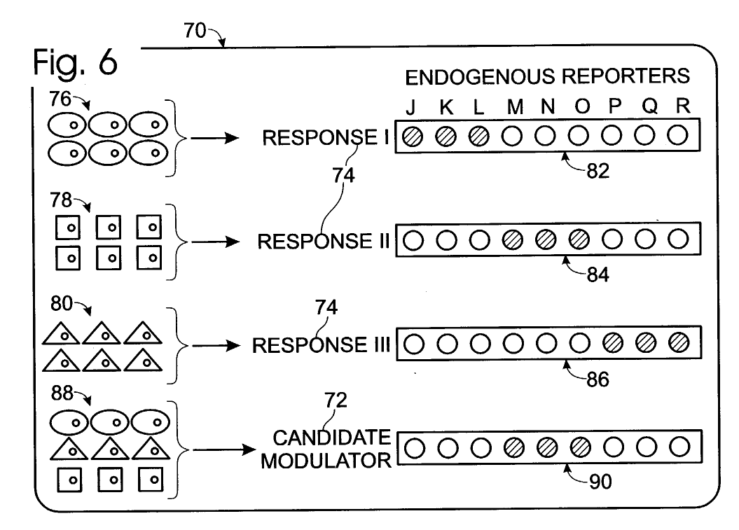

FIG. 6 shows a schematic view of another exemplary implementation 70 of method 10 of FIG. 1. Implementation 70 may compare the effect of a candidate modulator 72 to a plurality of cellular responses 74 produced in different cell types 76, 78, 80. Cell types 76-80 may be analyzed separately to identify endogenous reporter genes that are selective for each response, as shown at 82, 84, and 86, respectively. These cells types then may be provided as a mixture 88 to test the effect of candidate modulator 72 on the reporter genes. Here, modulator 72 selectively changes the expression of genes M-O, shown at 90. Accordingly, modulator 72 may be a selective agonist of response II. In other embodiments, responses I-III may be initiated (or at least initiating conditions, such as ligands, provided) and the mixture exposed to modulator 72 to test for antagonist or agonist activity of the modulator.

### Example 2

This example presents an alternative description of selected aspects of the invention.

Mother nature has evolved many “specific” transcripts that are generated when certain receptors are activated. Such specific transcripts may be identified through expression profiling on thousands of genes using microarrays. Each receptor of interest is activated with its corresponding ligand and then the expression profile is examined. Endogenous genes (could be one or more) that are specifically up (or down) regulated in response to the ligands are identified. If it is difficult to find receptor-specific target genes, one could look at a different cell line. The only cellular requirements are that the cells express the receptor of interest and that all the cell lines used are able to be co-cultured in the “multiplexed” environment long enough for the experiment to take place. In some embodiments, it may be possible to find a single cell type that expresses five receptors of interest, each of which determines a distinct RNA expression profile. Therefore, the tissue culture load would be minimal, and coded carriers would not be required. Instead, the cells would be grown, and specific RNA expression measured. The decoding is the same process as the data collection. The “code” is the unique RNA that is providing the data. A given profile (data/code) for a receptor could involve many RNAs. Therefore, with the standardization of these to internal controls, the measurements could be much more robust.

### Example 3

This example describes selected embodiments of the invention, presented as a series of indexed paragraphs.

1. A method for multiplexed analysis of ligand activity on a cell population, the cell population including a set of two or more predetermined endogenous reporter genes, where each gene of the set has an expression level that responds detectably upon exposure of the cell population to a corresponding known ligand, and expression of all other genes of the set is at least substantially unresponsive to the known ligand, the method comprising (1) exposing the cell population to a candidate ligand for sufficient time to produce a detectable response in the expression level, if any; and (2) measuring the detectable response, thereby relating the candidate ligand to a ligand-response pathway defined by the known ligand.

2. The method of paragraph 1, where the cell population is clonal or a mixed population of two or more distinct cell types.

3. The method of paragraph 2, where the detectable response of at least one gene of the set is produced by fewer than all of the distinct cells types.

4. The method of paragraph 1, where the cell population includes primary cells.

5. The method of paragraph 1, where the cell population expresses an exogenous receptor protein.

6. The method of paragraph 5, where the exogenous receptor protein is selected from the group consisting of cell-surface receptors and nuclear hormone receptors.

7. The method of paragraph 5, where the exogenous receptor protein is a G-protein coupled receptor (GPCR).

8. The method of paragraph 1, where the candidate ligand includes plural distinct candidate ligands.

9. The method of paragraph 1, where the known ligand binds specifically to a cell-surface or nuclear receptor.

10. The method of paragraph 1, further comprising the step of identifying the endogenous reporter genes before the step of exposing by treating cells separately with each of the corresponding known ligands, the cells corresponding to at least a subset of the cell population.

11. The method of paragraph 10, where the step of identifying includes screening gene candidates using microarray hybridization.

12. The method of paragraph 1, where the step of exposing including combining the candidate ligand with each known ligand, so that the candidate ligand competes with each known ligand to define the detectable response.

13. The method of paragraph 1, where each of the known ligands alters the expression level from undetectable to detectable.

14. The method of paragraph 1, where the step of measuring comprises (1) isolating RNA from the cell population; (2) adding nucleic acid probes; and (3) treating the RNA and probes with a nuclease that degrades single-stranded RNA.

15. The method of paragraph 14, where the step of measuring includes hybridizing the treated RNA and probes to nucleic acids disposed on a solid support.

16. The method of paragraph 1, where the cell population includes at least one additional predetermined, endogenous reporter gene, the additional reporter gene responding detectably to the known ligand of only one gene of the set.

17. The method of paragraph 16, where the only one gene and the additional reporter gene each respond detectably in an at least substantially identical subset of cells in the cell population.

18. The method of paragraph 17, where the subset corresponds to one of plural distinct cell types in the cell population.

19. The method of paragraph 1, where the cell population expresses an exogenous protein.

20. The method of paragraph 19, where the exogenous protein is selected from the group consisting of kinases, phosphatases, DNA binding proteins, RNA binding proteins, methylases, demethylases, acetylases, deacetylases, methylases, demethylases, helicases, topoisomerases, transcription factors, transcription cofactors, and transcription inhibitors.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

I claim:

1. A method of performing a multiplexed experiment on cells, the cells including an endogenous reporter gene for each of a plurality of responses, the endogenous reporter gene having a level of expression that changes substantially for only one of the responses, the method comprising:
exposing the cells to a candidate modulator; and
measuring the level of expression of the endogenous reporter gene for each of the responses to determine if the candidate modulator changes the level, thereby relating the candidate modulator to each of the responses.

2. The method of claim 1, wherein the cells include at least two different cell types, at least one of the responses being at least substantially restricted to less than all of the at least two different cell types.

3. The method of claim 2, each response being at least substantially restricted to one of the cell types.

4. The method of claim 1, wherein the cells includes primary cells.

5. The method of claim 1, wherein the cells express an exogenous receptor protein.

6. The method of claim 5, where the exogenous receptor protein is selected from the group consisting of cell-surface receptors and nuclear hormone receptors.

7. The method of claim 6, wherein the exogenous receptor protein is a G-protein coupled receptor.

8. The method of claim 1, wherein each of the responses is produced by contacting the cells with a different ligand.

9. The method of claim 8, wherein the different ligand binds specifically to a cell-surface receptor or a nuclear receptor.

10. The method of claim 1, further comprising the step of identifying the endogenous reporter gene for each response, before the step of exposing, by separately producing each response in a cell population.

11. The method of claim 10, wherein the step of identifying includes screening candidate genes using microarray hybridization.

12. The method of claim 1, wherein each response increases the level of expression of the corresponding reporter gene.

13. The method of claim 1, the endogenous reporter gene for each response being a plurality of genes.

14. The method of claim 1, wherein the step of measuring comprises
isolating RNA from the cells;
adding nucleic acid probes; and
treating the RNA and probes with a nuclease that degrades single-stranded RNA.

15. The method of claim 14, wherein the step of measuring includes hybridizing the treated RNA and probes to nucleic acids disposed on a solid support.

16. The method of claim 1, wherein the steps of exposing and measuring are performed a plurality of times in different wells of a microplate, the candidate modulator being different each of the times.

17. A method of performing a multiplexed experiment on cells, the cells including an endogenous reporter gene for each of a plurality of responses to different ligands, the endogenous reporter gene having a level of expression that changes substantially for only one of the responses, the method comprising:
exposing the cells to a candidate modulator; and
measuring the level of expression of the endogenous reporter gene for each of the responses to determine if the candidate modulator changes the level substantially, thereby relating the candidate modulator to each of the responses.

18. The method of claim 17, wherein at least a portion of the step of exposing is conducted while all of the different ligands are present.

19. The method of claim 17, each of the different ligands binding to at least one of a nuclear hormone receptor and a G-protein coupled receptor.
